(19) United States
(12) Patent Application Publication
(10) Pub. No.: US 2011/0060269 A1
(43) Pub. Date: Mar. 10, 2011

(54) METHOD FOR KILLING CELLS USING PHOTOCATALYTIC TITANIUM DIOXIDE PARTICLES

(75) Inventors: Koki KANEHIRA, Kitakyushu-Shi (JP); Shuji SONEZAKI, Kitakyushu-Shi (JP); Yumi OGAMI, Yukuhashi-Shi (JP); Toshiaki BANZAI, Kitakyushu-Shi (JP); Junji KAMESHIMA, Fujisawa-Shi (JP)

(73) Assignee: TOTO LTD., Kitakyushu-Shi (JP)

(21) Appl. No.: 12/943,165

(22) Filed: Nov. 10, 2010

Related U.S. Application Data
(63) Continuation of application No. 11/883,212, filed on Jul. 3, 2008.

(30) Foreign Application Priority Data
Sep. 22, 2005 (JP) 2005-276706
Sep. 22, 2005 (JP) 2005-276707

(51) Int. Cl. A61M 37/00

(52) U.S. Cl. 604/20

(57) ABSTRACT

Photocatalytic titanium dioxide particles are disclosed having improved dispersibility into an aqueous solvent not only under neutral physiological conditions in vivo but also over a wide pH range, and improved cell affinity and cell uptake property. The photocatalytic titanium dioxide particles comprise particles comprising photocatalytic titanium dioxide and a cationic hydrophilic polymer modifying surfaces of the photocatalytic titanium dioxide particles, wherein the hydrophilic polymer is bonded to the photocatalytic titanium dioxide particles. The particles are very useful for medical applications, such as destruction of cancer cells, e.g., when administered to a mammal the titanium dioxide particles are taken up by cells of the mammal, and if then irradiated with UV light the particles kill the cells via photocatalytic degrading capability.

### CROSS-REFERENCE TO RELATED APPLICATIONS

The present application is a continuation of U.S. patent application Ser. No. 11/883,212, filed Jul. 3, 2008, which is the U.S. National phase of, and claims priority based on PCT/JP2006/306179, filed 27 Mar. 2006, which, in turn, claims priority from Japanese patent applications 2005-276706 and 2005-276706, both filed 22 Sep. 2005. The entire disclosure of each of the referenced priority documents is incorporated herein by reference.

### BACKGROUND OF THE INVENTION

1. Field of the Invention

The present invention relates to photocatalytic titanium dioxide particles surface-modified with a cationic hydrophilic polymer, a dispersion thereof, and a process for producing the same. The present invention also relates to a dispersion containing photocatalytic titanium dioxide composite particles which can degrade cancer cells, endocrine disrupting chemicals and the like by immobilizing onto the particles a biopolymer, such as an antibody capable of recognizing a molecule of the cancer cells, endocrine disrupting chemicals and the like, and then exposing the particles to ultraviolet ray; and a process for producing the same.

2. Background Art

It is known that titanium dioxide has high photocatalytic degrading capability, high chemical stability even in the air or solutions, and non-toxicity and safety in light shielded animal bodies. In view of these, application of titanium dioxide to medical fields have been studied. Japanese Patent Laid-Open Publication No. 316946/2002, Japanese Patent Laid-Open Publication No. 316950/2002, and R. Cai et al.: Cancer Research, 52, 2346-2348 (1992) propose cancer treatments with titanium dioxide. These treatments are intended to kill cancer cells by shooting into target cancer cells metal particles such as gold particles supporting titanium dioxide and then by irradiating light such as ultraviolet ray to the metal particles. In particular, titanium dioxide can control ON/OFF of a chemical reaction switch, reaction region, and reaction strength, by taking advantage of light. Accordingly, titanium dioxide has been considered effective for the establishment of a treatment method utilizing a site-specific control mechanism.

Titanium dioxide has been said to have an isoelectric point around pH 6. For this reason, titanium dioxide particles are disadvantageously aggregated in an aqueous solvent having a substantially neutral pH value, and it was very difficult to disperse the titanium dioxide particles homogeneously. Accordingly, various studies have been made to homogeneously disperse the titanium dioxide particles in an aqueous dispersion medium. For example, Barbe Christophe et al.: Journal of the American Ceramics Society, 80, 3157-3171 (1997) and Vorkapic Danijela et al.: Journal of the American Ceramics Society, 81, 2815-2820 (1998) propose titanium dioxide sols acidified with nitric acid produced by precipitating titanium hydroxide from titanium isopropoxide and then peptizing the titanium hydroxide under acid conditions with nitric acid at an elevated temperature. Further, Japanese Patent Laid-Open Publication No. 67516/1998 proposes a method comprising the steps of adding an aqueous ammonia to an aqueous titanium tetrachloride solution to form titanium hydroxide precipitates, adding an aqueous hydrogen peroxide to the precipitates to allow a reaction at 100° C. for 6 hr, so as to form a sol of titanium dioxide particles having surfaces modified with a peroxo group. Japanese Patent Laid-Open Publication No. 319577/1999 proposes a process for producing a dispersion of composite titanium dioxide particles comprising the steps of coating the surfaces of titanium dioxide particles with a porous silica and dispersing the coated particles under alkaline conditions for stabilization. Japanese Patent Laid-Open Publication No. 212315/1990 proposes a process for producing an aqueous titanium dioxide solution having dispersibility enhanced by incorporating polycarboxylic acid or its salt as a dispersing agent. In these techniques, however, the titanium dioxide particles are disadvantageously aggregated under substantially neutral physiological conditions to damage biologic bodies and thus create unfavorable conditions. Accordingly, it has been difficult to apply the titanium dioxide particles as a medical material to biologic bodies.

WO 2004/087577 discloses that good dispersibility in a substantially neutral state can be realized by surface-modified titanium oxide particles produced by ester-bonding a hydrophilic polymer such as polyacrylic acid to titanium oxide particles through a carboxyl group. This technique aims at use of an anionic polymer such as polyacrylic acid.

In recent years, cationic liposome has widely been used as a carrier for nonvirus introduction of a gene into cells. The cationic liposome is a vesicle which comprises a phospholipid being a constituent of a biomembrane and contains a positive charge functional group such as a quaternary amine on the surface of a liposome membrane. Since the liposome is a vesicle having excellent biocompatibility so as to enable various drugs to be encapsulated in the vesicle, the liposome has been utilized as carriers for drugs. Further, providing the outer surface of the liposome with positive charges can enhance interaction between the liposome and negatively charged cells, enabling a drug to be encapsulated into the cells. Liposomes are morphologically classified into small single-membrane liposomes, large single-membrane liposomes, and multilayered liposomes. Yoshida J et al., Jpn J Cancer Res., 87, 1179-1183 (1996) discloses a method of encapsulating magnetites, which are iron oxide Fe₃O₄ particles having a diameter of about 10 nm, into the cationic liposome of this composition, and an attempt to target tumors through a local administration. In this method, a solution containing the above-mentioned lipid dissolved in chloroform is removed by evaporation by a rotary evaporator, and the lipid film thus formed is dried under reduced pressure. Magnetite is then added to the dried film, followed by vortex treatment and ultrasonication to form a magnetite cationic liposome (MCL). The introduction efficiency of MCL into T-9 rat glioma cells in vitro has been reported to be higher than a magnetite encapsulated into a charge-free neutral liposome (magnetoliposome) by a factor of at least 10. However, encapsulating such particles into the liposomes increases the size larger than the original particle size by a few dozen times. Further, due to a complicated liposome preparation process, it is considered difficult to provide homogeneous products.

### SUMMARY OF THE INVENTION

The inventors have now found that chemically binding a cationic hydrophilic polymer onto the surface of photocatalytic titanium dioxide particles for surface modification significantly improves dispersibility of the photocatalytic titanium dioxide particles into an aqueous solvent not only under neutral physiological conditions in vivo but also over a wide pH range, and also improves cell affinity and cell uptake property of the photocatalytic titanium dioxide particles, so as to render photocatalytic titanium dioxide particles very useful for medical applications, such as destruction of cancer cells.

It is therefore an object of the present invention to provide photocatalytic titanium dioxide particles having improved dispersibility into an aqueous solvent not only under neutral physiological conditions in vivo but also over a wide pH range, and improved cell affinity and cell uptake property, so as to be very useful for medical applications, such as destruction of cancer cells, and a process for producing the same.

According to an aspect of the present invention, there is provided photocatalytic titanium dioxide particles comprising:

particles comprising photocatalytic titanium dioxide and

a cationic hydrophilic polymer modifying surfaces of the photocatalytic titanium dioxide particles,

wherein the hydrophilic polymer is bonded to the photocatalytic titanium dioxide.

According to another aspect of the present invention, there is provided a process for producing the photocatalytic titanium dioxide particles, the process comprising:

a first step of dispersing a titanium dioxide sol in a solvent to obtain a dispersion;

a second step of dispersing a cationic hydrophilic polymer in a solvent to obtain another dispersion;

a third step of mixing both the dispersions together to obtain a mixed dispersion;

a fourth step of heating the mixed dispersion;

a fifth step of separating the photocatalytic titanium dioxide particles from the hydrophilic polymer remaining unbonded; and

a sixth step of purifying the photocatalytic titanium dioxide particles.

### BRIEF DESCRIPTION OF THE DRAWINGS

FIG. 1 is a schematic view showing a photocatalytic titanium dioxide particle according to the present invention.

FIG. 2 is a schematic view showing a dispersion containing a photocatalytic titanium dioxide composite particle according to the present invention.

FIG. 3 is a graph showing measurement results of the photocatalytic activity (indicated as reduction in absorbance caused by the decomposition of methylene blue) of photocatalytic titanium dioxide particles according to the present invention, wherein white circles represents data without ultraviolet irradiation while black circles represents data with ultraviolet irradiation, when polyethyleneimine-bonded titanium dioxide particles (anatase-type) prepared in Example A1 were used.

FIG. 4 is a graph showing measurement results of the average dispersed particle diameter of photocatalytic titanium dioxide particles according to the present invention at each pH value.

FIG. 5 is a graph showing measurement results of the average dispersed particle diameter of photocatalytic titanium dioxide particles according to the present invention in each salt concentration.

FIG. 6 is a photograph showing observation results of homogeneity (transparency) of photocatalytic titanium dioxide particles according to the present invention.

FIG. 7 is a graph showing measurement results of the cytotoxity in each concentration of photocatalytic titanium dioxide particles according to the present invention.

FIG. 8 is a photograph showing observation results of the cell uptake property of photocatalytic titanium dioxide particles according to the present invention.

FIG. 9 is a graph showing results of aggregation (indicated as increase in absorbance) of streptavidin-immobilized photocatalytic titanium dioxide composite particles according to the present invention with a biotin dimer.

FIG. 10 is a photograph showing observation results of homogeneity (transparency) of a dispersion containing photocatalytic titanium dioxide composite particles according to the present invention.

### DETAILED DESCRIPTION OF THE INVENTION

### Photocatalytic Titanium Dioxide Particles

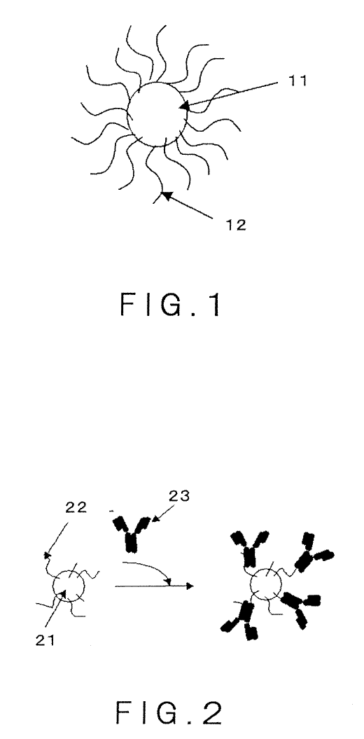

The photocatalytic titanium dioxide particles according to the present invention comprise particles comprising photocatalytic titanium dioxide and a cationic hydrophilic polymer modifying surfaces of the photocatalytic titanium dioxide particles. The hydrophilic polymer is bonded to the photocatalytic titanium dioxide. FIG. 1 is a schematic view showing a photocatalytic titanium dioxide particle according to an aspect of the present invention. As shown in FIG. 1, the photocatalytic titanium dioxide particle according to the present invention comprises a cationic hydrophilic polymer 12 on the surface of a photocatalytic titanium dioxide particle 11. The cationic polymer is bonded to the titanium dioxide, enabling the photocatalytic titanium dioxide particles to be stably dispersed into an aqueous solution without adding other substances such as a dispersant.

Specifically, the photocatalytic titanium dioxide particles according to the present invention have a cationic hydrophilic polymer bonded onto their surfaces, charging the photocatalytic titanium dioxide particles positively at a substantially neutral pH value. This causes electric repulsion among the particles, leading to satisfactory dispersibility without aggregation in aqueous solvents which are approximately neutral or have a wide pH range. Further, by virtue of the above properties, the dispersion of photocatalytic titanium dioxide particles according to the present invention can utilize water- or salt-containing buffer solutions having various pH values as a solvent, realizing satisfactory dispersibility without adding other substances such as a dispersant under physiological conditions, so as to maintain in a stably dispersed state over 24 hr or more. In addition, the photocatalytic titanium dioxide particles are positively charged so as to be able to capture a negatively charged substance and then to strongly degrade a target substance upon exposure to ultraviolet ray or the like. Since the surface of the cells is negatively charged in general, the photocatalytic titanium dioxide particles have a high level of cell affinity and cell uptake property. This enables the photocatalytic titanium dioxide particles to be particularly applied to medical applications, such as cancer cell destruction.

According to a preferred embodiment of the present invention, the hydrophilic polymer may be a hydrophilic polymeric amine. Since the amine and titanium dioxide are strongly bonded to each other, the photocatalytic titanium dioxide particles can be more stably dispersed into an aqueous solution. Further, the isoelectric point of the amine contained in the hydrophilic polymer affects the isoelectric point of the titanium dioxide particles, causing electrical repulsion among the particles even in a neutral aqueous solvent so as to provide satisfactory dispersibility.

According to a preferred embodiment of the present invention, any of crystalline form between anatase and rutile is usable as a material for photocatalytic titanium dioxide particles. This is because these titanium dioxides have the same chemical property of producing a hydroxyl group through hydration in spite of the difference in crystalline form, enabling a cationic hydrophilic polymer to be bonded to the these titanium dioxides for attaining surface modification. Anatase-type is suitable for a strong photocatalytic activity, while rutile-type is suitable for properties such as high refractive index as in cosmetics. For the same reason, composite titanium dioxide particles comprising titanium dioxide and a magnetic material are suitable as well as simple titanium dioxide particles. The dispersed particle diameter is preferably 2 to 200 nm in view of flexibility in type of usage. This is because a particle diameter of more than 200 nm leads to an increase in gravity on the particles, facilitating the settlement of the particles.

According to a preferred embodiment of the present invention, the photocatalytic titanium dioxide particles preferably have a dispersed particle diameter of 2 to 500 nm in view of dispersibility. In applications into living bodies for cancer treatments, it is more preferred that the dispersed particle diameter be 50 to 200 nm for effective accumulation into tumor cells. A dispersed particle diameter in the above range enables stable dispersion under physiological conditions for 24 hr or more. The term “dispersed particle diameter” as used herein refers to an average value calculated by a cumulant analysis after a measurement by a dynamic light scattering method. The expression “under physiological conditions” as used herein refers to “in the presence of a phosphate buffer brine (pH 7.4), which has a composition comprising 137 mM NaCl, 8.1 mM Na₂HPO₄, 2.68 mM KCl, and 1.47 mM KH₂PO₄, at 25° C. and 1 atm.

According to a preferred embodiment of the present invention, the hydrophilic polymer is preferably a hydrosoluble polymer because the present invention aims at using the photocatalytic titanium dioxide particles as a dispersion in an aqueous solution. Any hydrosoluble polymer can be used which can be strongly bonded to titanium dioxide and is an amine having an average molecular weight of 1000 to 100000. Examples of such a hydrosoluble polymer include polyamino acids, polypeptides, polyamines, and copolymers having a plurality of amine units in the molecule thereof. Specifically, it is more preferable to use polyamines such as polyethyleneimine, polyvinylamine and polyallylamine in view of hydrolyzability and solubility of the hydrosoluble polymer. More specifically, basic polyamino acids such as polyornithine and polylysine may be used so that the amine and/or carboxyl groups in the polymer can be strongly bonded to titanium dioxide to form desired photocatalytic titanium dioxide particles.

According to a preferred embodiment of the present invention, the surface potential of the photocatalytic titanium dioxide particles is preferably +20 mV or more for good dispersibility and cell uptake property, more preferably +40 mV or more regarded generally as a potential which can realize satisfactory self-dispersion (a state that the particles are not precipitated).

### Dispersion

According to a preferred embodiment of the present invention, a dispersion of photocatalytic titanium dioxide particles is provided comprising photocatalytic titanium dioxide particles and an aqueous solvent in which the photocatalytic titanium dioxide particles are dispersed. In the aqueous dispersion medium, electric repulsive force acts on among particles by positive charges (preferably positive charges by the amine) present on the surface of the photocatalytic titanium dioxide particles, enabling the particles to exist stably without causing aggregation over a long period of time. In addition, the particles are basically very stable even when the pH value is varied or when an inorganic salt is added. When the hydrophilic polymer is a hydrophilic polymeric amine, the particles can provide extremely good dispersibility in an aqueous dispersion medium having a pH value of 3 to 9 because the isoelectric point of the photocatalytic titanium dioxide particles reflects the isoelectric point of the hydrophilic polymeric amine, leading to an increase in electric repulsive force acted on among the particles with reducing the pH value in an aqueous dispersion medium having a pH value of 9 or lower. Accordingly, it is possible to use a pH buffer solution as the aqueous solvent for the dispersion of the present invention. That is, the photocatalytic titanium dioxide particles according to the present invention provide good dispersibility as far as the pH is in the range of 3 to 9 even when any buffer component is contained in the aqueous dispersion medium. Preferred examples of the buffer solutions include glycine buffer solutions, acetate buffer solutions, phosphate buffer solutions (including PBS), carbonate buffer solutions, McIlvaine buffer solutions, Good's buffer solutions, and borate buffer solutions. The fact that buffer solutions having an approximately neutral pH value are usable means that the photocatalytic titanium dioxide particles are extremely advantageous in applications to biotechnical fields and pharmaceutical and medical fields. In order to maintain the good dispersibility, the amino group/titanium dioxide amount ratio (mol/g) in the dispersion of the surface-modified titanium dioxide particles may vary depending upon reaction conditions but is preferably approximately not less than 1.5×10⁻².

According to a preferred embodiment of the present invention, the salt concentration of the aqueous solvent is preferably 1 M or less. A concentration of the dispersion around this level enables the dispersion to be kept stable without agglomeration over at least 24 hr or longer due to the electric repulsive force among the photocatalytic titanium dioxide particles. The salt concentration is more preferably about 100 mM to 300 mM. A salt concentration within this range enables the photocatalytic titanium dioxide particles to exist stably in a dispersed state even under neutral physiological conditions in vivo.

According to a preferred embodiment of the present invention, the dispersion of the photocatalytic titanium dioxide particles preferably comprises not more than 20% by weight of the photocatalytic titanium dioxide particles. A dispersion with a concentration of this level enables the dispersion to be kept stable without agglomeration over at least 24 hr or longer due to electric repulsive force among the photocatalytic titanium dioxide particles. The photocatalytic titanium dioxide particle content is more preferably 0.0001 to 0.1% by weight. The content of this level leads to improved safety to cells in applications to living bodies to be considered.

In view of the above, it is possible to provide the photocatalytic titanium dioxide particle-containing dispersion according to the present invention as homogeneous and stable dispersions comprising water, various pH buffer solutions, transfusions, or physiological salines. It is also possible to produce ointments, spray preparations, or the like comprising the photocatalytic titanium dioxide particles according to the present invention. In addition, a medical treatment can be conducted by applying an ointment or spray preparation comprising a dispersion containing photocatalytic titanium dioxide particles of the present invention directly to an affected part such as skin and then exposing the part to sunlight, ultraviolet ray, or the like.

Although light source device for exciting and activating the photocatalytic titanium dioxide particles does not have to be a special device, the wavelength to be used is preferably not more than 400 nm in view of the band-gap of titanium dioxide. In external applications as in skin or the like, sunlight, conventional ultraviolet lamps, or black lights can be preferably used. When the affected part is located within the body, an ultraviolet light fiber may be mounted on an endoscope to emit ultraviolet ray. In view of a phototherapy in which ultraviolet ray particularly with a wavelength around 280 nm is applied topically to the affected part to destroy the lesion part, a dispersion containing titanium dioxide composite particles according to the present invention can also be applied as an action enhancer.

The photocatalytic titanium dioxide particles according to the present invention have positive surface charge due to the presence of amine on the surface, leading to extremely high affinity and cell uptake property to cells generally having negative surface charge, so that the contact of the photocatalytic titanium dioxide particles of the present invention with the cells immediately initiates bonding and uptaking to the cells. It is therefore very effective to apply the photocatalytic titanium dioxide particles particularly to the surface of the skin of the living body, surface layer parts in the living body such as of trachea and digestive organs, and various affected parts present in the living body. For example, a medical treatment can be conducted by using sunlight, an ultraviolet lamp, or a light source for use in medical applications, after an ointment or spray preparation comprising the photocatalytic titanium dioxide particle-containing dispersion of the present invention is applied directly to a part affected by a cancer such as skin cancer or pharyngeal cancer or is administered topically to a solid cancer by injection. In this case, the light or ray can be irradiated through an endoscope, achieving a high therapeutic effect with a simple procedure.

Dispersion of Photocatalytic Titanium Dioxide Composite Particles with Biomolecules Immobilized Thereon

According to a preferred embodiment of the present invention, the dispersion is preferably a dispersion containing photocatalytic titanium dioxide particles comprising a biomolecule immobilized on the hydrophilic polymeric amine. Specifically, the dispersion according to this embodiment comprises a hydrophilic polymeric amine on the surface of titanium dioxide where the hydrophilic polymeric amine is strongly bonded to the titanium dioxide, while enabling the biomolecule to be immobilized onto the hydrophilic polymeric amine. This dispersion also has satisfactory dispersibility and is stable with no addition of any other material such as a dispersant. This leads to simultaneous attainment of a selective adsorbability and a photocatalytic activity in photocatalytic titanium dioxide composite particles prepared by modifying the surface of titanium dioxide with a hydrophilic polymer and then immobilizing a biomolecule onto the hydrophilic polymer. In view of these, according to this embodiment, stable dispersion and presence can be realized even under neutral physiological conditions in a living body having a selective adsorbability. In this embodiment, in order to preferably attain both of selective adsorbability and photocatalytic activity, photocatalytic titanium dioxide is preferably present on at least a part of the surface of the photocatalytic titanium dioxide particle, while the photocatalytic titanium dioxide is preferably anatase-type titanium dioxide superior in photocatalytic activity. The aqueous solvent is preferably an aqueous solution which is allowed to be introduced into the living body.

FIG. 2 schematically shows a dispersion containing a photocatalytic titanium dioxide composite particle with a biomolecule immobilized thereon according to this embodiment. The dispersion comprising photocatalytic titanium dioxide composite particles according to the present invention is obtained by dispersing anatase-type titanium dioxide 21 and a hydrophilic polymeric amine 22 to be bonded to a biomolecule, into an aprotic polar solvent, allowing the mixture to react at 90 to 180° C. for 1 to 12 hr to form a bond between the hydrophilic polymer and titanium dioxide, then dispersing the reaction product into an aqueous solution, and immobilizing a biomolecule 23 to the hydrophilic polymeric amine. This enables the photocatalytic titanium dioxide composite particles according to the present invention to be stably dispersed into an aqueous solution with no addition of any other material such as a dispersant. For the purpose of immobilizing the biomolecule, a free amine may be used which is not involved in bonding between titanium dioxide and the hydrophilic polymer on the surface of the photocatalytic titanium dioxide composite particles. Since an aldehyde group or the like derived from an amino group, a carboxyl group, a thiol group, or a sugar chain is present on the biomolecular side, the amine and the biomolecule can be covalently bonded with the aid of a suitable crosslinking agent.

A wide variety of biomolecules are considered to be usable in the present invention, including proteins as the most promising biomolecule. According to the present invention, biomolecules ranging from antibodies and receptors to low-molecular peptides, as proteins, can be suitably immobilized. In view of chemical composition of the protein, amino group, carboxyl group, or thiol group can be a target functional group for immobilizing the protein to photocatalytic titanium dioxide composite particles, while aldehyde group can be a target functional group for immobilizing a sugar protein. Further, the biomolecule and the photocatalytic titanium dioxide composite particles can be immobilized to each other through interaction between biotin and avidin.

Examples of preferred biomolecules include amino acids, peptides and simple proteins (such as lectin), and conjugated proteins; nucleosides, nucleotides, and nucleic acids; monosaccharides, sugar chains, polysaccharides, and complex carbohydrates; simple lipids, complex lipids, and liposomes; and combinations thereof.

According to a preferred embodiment of the present invention, bonding between the photocatalytic titanium dioxide composite particle and the biomolecule can be achieved by using a bifunctional linker reagent. With the use of a bifunctional linker reagent having a homo functional group, a covalent bond can easily be introduced into between the amine on the surface of the photocatalytic titanium dioxide composite particle and the amino group derived from the biomolecule. On the other hand, with the use of a bifunctional linker reagent having a hetero functional group, a biomolecule having thiol or carboxyl group can be introduced.

Further, not only the biomolecule but also a fluorescent dye, a detection probe substance, or the like can be immobilized onto the photocatalytic titanium dioxide composite particles through the introduction of a suitable functional group.

Examples of preferably usable homo linker reagents for bonding amino groups to each other include those containing N-hydroxysuccinimido ester such as disuccinimidyl glutarate and bis(sulfosuccinimidyl) suberatate; and those containing imide ester such as dimethyl adpimidate and dimethyl suberimidate.

Regarding combinations of the hetero functional groups include, the above N-hydroxysuccinimido ester or imidoester can be used for the amine on the surface of the photocatalytic titanium dioxide composite particle, while a substance containing maleimido group such as N-(ε-maleimidocaproyloxy)succinimide ester and the like can be used for thiol group on the biological substance side.

A nucleic acid can be immobilized onto the photocatalytic titanium dioxide composite particles in the same manner by synthesizing a modified DNA with use of an amination primer, a thiolation primer, or a biotinylation primer in the DNA amplification through a polymerase chain reaction (PCR). For example, when an amination DNA is used in the immobilization, the use of a bifunctional homo linker for bonding between the nucleic acid and the amine on the surface of the photocatalytic titanium dioxide composite particle can realize immobilization by simply mixing the two materials together. When a thiolation DNA is used, the use of the above bifunctional hetero linker can realize amine-thiol bonding. When a biotinylation DNA is used, streptavidin should be introduced into the photocatalytic titanium dioxide composite particle, while streptavidin can easily be introduced by using the bifunctional homo linker for the amino group.

A sugar chain derived from a conjugated protein or saccharide can be immobilized by oxidizing cis-diol with periodic acid or the like to aldehyde and forming a Schiff's base in the presence of the amine in the photocatalytic titanium dioxide composite particle and sodium cyanoborohydride. Alternatively, crosslinking may also be carried out by using a bifunctional linker.

When a carboxyl group is present on the biomolecular side such as a part of protein or saccharide, the biomolecule and the photocatalytic titanium dioxide particle can be crosslinked by causing activation with 1-ethyl-3-(3-dimethylaminopropyl)carbodiimide (EDC) and mixing the biomolecule and the photocatalytic titanium dioxide composite particles together.

The photocatalytic titanium dioxide composite particles in the dispersion according to the present invention have positive surface charge due to the presence of amine on the surfaces of the particles, resulting in an extremely high level of cell affinity and cell uptake property so that the photocatalytic titanium dioxide composite particles of the present invention can be bonded to or uptaken by cells immediately upon contact. This renders the application of the photocatalytic titanium dioxide composite particles particularly to the surface of the skin in the living body, the surface layer part in the living body such as trachea and digestive organs, and various affected parts present in the living body very effective, and enables localization of the photocatalytic titanium dioxide composite particles in cancer cells. For example, bonding of a signal for migration into the nucleus facilitates approach to DNA within the nucleus, realizing a higher level of therapeutic effect. For example, a medical treatment can be conducted with sunlight, ultraviolet lamps, light sources for use in medical applications, and the like, after applying an ointment or spray preparation containing the photocatalytic titanium dioxide particle-containing dispersion of the present invention directly to a part affected by cancer such as skin cancer or pharyngeal cancer or, in the alternative, after administering the photocatalytic titanium dioxide particle-containing dispersion topically to a solid cancer by injection. In this case, light irradiation may be carried out through an endoscope, achieving a high level of therapeutic effect with a simple method.

In particular, in the photocatalytic titanium dioxide composite particle-containing dispersion according to the present invention, a biomolecule, such as proteins, antibodies, and DNAs, capable of recognizing a molecular, such as cancer cells, endocrine disrupting chemicals, and the like, can be immobilized onto an anatase titanium dioxide modified with a hydrosoluble polymer, so as to be able to recognize these molecules and to provide reactions degrading these substances through photocatalytic action upon exposure to ultraviolet ray or the like. The photocatalytic titanium dioxide composite particles contained in the dispersion according to the present invention can specifically recognize and capture a target substance in water or an aqueous solution and can strongly degrade the target substance upon exposure to ultraviolet light or the like. In particular, usability in an aqueous medium, capability of accurately capturing a target substance, and possessing a strong photocatalytic activity are very advantageous for medical applications, such as those for degrading endocrine disrupting chemicals or destroying cancer cells.

### Production Process

According to a preferred embodiment of the present invention, the photocatalytic titanium dioxide particles of the present invention can be produced through a reaction for bonding a hydrophilic polymer to the surfaces of photocatalytic titanium dioxide particles by a process comprising the steps of (1) dispersing a titanium dioxide sol in a solvent to obtain a dispersion; (2) dispersing a cationic hydrophilic polymer in a solvent to obtain another dispersion; (3) mixing these dispersion; (4) heating the mixed dispersion; (5) separating the photocatalytic titanium dioxide particles from the hydrophilic polymer remaining unbonded; and (6) purifying the photocatalytic titanium dioxide particles.

The titanium dioxide sol used in the present invention may be synthesized by using titanium tetraisopropoxide or the like as a raw material, while a conventional acid titanium dioxide sol peptitized with an inorganic acid may be also used. On the other hand, a solvent which can dissolve both the titanium dioxide sol and the hydrophilic polymer is suitable for use in the steps (1) and (2). This is because, agglomeration of titanium dioxide in the solvent leads to a decrease in surface area where a bonding reaction can take place between titanium dioxide and the hydrophilic polymer, increasing the dispersed particle diameter in the aqueous solvent after the reaction to deteriorate the dispersibility. In this case, a solvent reactive with the surfaces of the titanium dioxide particles is unsuitable. In particular, alcohols containing hydroxyl group inhibit a bonding reaction with an aimed hydrophilic polymer due to formation of ether bonds with the surfaces of the titanium dioxide particles upon heating. In this case, the surface properties of the titanium dioxide particles depend upon the properties of alcohol used, and the dispersibility of the titanium dioxide particles in an aqueous dispersion medium is significantly lowered. The solvent to be used in the present invention is preferably an aprotic polar solvent in view of the reactivity. Specifically, dimethylformamide, dioxane, or dimethylsulfoxide may be used as the solvent, with diemthylformamide being more preferred for use as a solvent in view of volatility. Through a reaction under such conditions, titanium dioxide can be chemically bonded to the hydrophilic polymer, enabling a high level of dispersion stability.

In the step (3), the titanium dioxide dispersion in the above solvent and the hydrophilic polymer dispersion are then mixed together to be stirred, so as to form a dispersion in which titanium dioxide and the hydrophilic polymer are homogeneously dispersed. In this case, it is desirable that each of the dispersions is prepared before being mixed since adding the hydrophilic polymer directly into the titanium dioxide dispersion may causes agglomeration of titanium dioxide.

In the step (4), this mixture is then heated for a bonding reaction, which can proceed without applying any pressure if a proper ratio between the titanium dioxide and the hydrophilic polymer is selected. A reaction is, however, desirably conducted under a pressure being applied because the applied pressure further accelerates the reaction. In this case, when polyethyleneimine (average molecular weight: 10000) is used as the hydrophilic polymer, the final concentration of polyethyleneimine is preferably brought to not less than 10 mg/ml for better dispersibility. The production process of the present invention is characterized by the heating temperature of 80 to 220° C. A heating temperature below 80° C. reduces the amount of the hydrophilic polymer participating in the bonding, deteriorating dispersibility in the aqueous solvent. When the reaction is carried out under a pressure being applied, a heating temperature above 220° C. is unsuitable due to a problem with sealability of reaction vessels. Further, when the reaction is conducted at a temperature at or above the boiling point of water, it is preferred to allow the reaction to proceed under a pressure being applied since the complete volatilization of the water contained in the titanium dioxide sol out of the reaction system leads to agglomeration of titanium dioxide. Water content in the reaction solution can be different depending on reaction conditions but is preferably not more than about 4% since an excessively high water content in the reaction solution may inhibit the reaction.

In the step (5), the photocatalytic titanium dioxide particles thus produced are separated from the hydrophilic polymer remaining unbonded. Means for separation, such as dialysis, ultrafiltration, gel filtration chromatography, and sedimentation, are suitably used. In the separation through dialysis or ultrafiltration, a dialysis membrane or an ultrafiltration membrane should be used corresponding to the molecular weight of the hydrophilic molecule used. Although the separation can be carried out in accordance with any of the above methods, an organic solvent precipitation method using an organic solvent is preferably employed in view of operation simplicity.

When an organic solvent precipitation method is utilized, isopropanol is added, after the completion of the reaction, to the reaction solution in an amount of twice the amount of the reaction solution, and the mixture is then allowed to stand at room temperature for 30 min. The addition of a suitable amount of isopropanol causes precipitation of the particles due to a decrease in solubility. While the hydrophilic polymer not bonded to the particles stays in the solution without agglomeration, the hydrophilic polymer remaining unbonded can be removed by centrifuging this solution. The collected precipitate is washed with 70% ethanol, and the washings are removed by centrifugation.

In the step (6), the precipitate of the photocatalytic titanium dioxide particles are then suspended in an aqueous solvent having a pH value of 3 to 9, more preferably a pH value of 5 to 8. In this case, water, desired pH buffer solutions, and the like are suitable as the aqueous solvent. A dried powder of photocatalytic titanium dioxide particles can be produced by stirring this suspension or exposing this suspension to ultrasonic waves to homogeneously disperse the surface modified titanium dioxide particles, desalting the dispersion, and drying the dispersion. The facts that the handling is simple and stable powder can be produced are very advantageous for use of the photocatalytic titanium dioxide particles in various applications.

Further, the same production process and purification method as described above can be also applied in the case of a composite titanium dioxide particles composed of titanium dioxide and a magnetic material, since the properties of the particles in the solvent are similar to those of the titanium dioxide per se as far as titanium dioxide is exposed on the surface of the particle. These photocatalytic titanium dioxide particles are very useful since they have magnetism, which enables the particles to be easily recovered with a magnet after the particles are applied, for example, to degradation treatment of harmful substances in water.

### EXAMPLES

### Example A1

### Introduction of Polyethyleneimine into Titanium Dioxide Particles (1)

Titanium tetraisopropoxide (3.6 g) was mixed with 3.6 g of isopropanol, and the mixture was added dropwise to 60 ml of ultrapure water under ice cooling for hydrolysis. After the dropwise addition, the mixture was stirred at room temperature for 30 min. After the stirring, 1 ml of 12 N nitric acid was added dropwise to the mixture, and the mixture was stirred at 80° C. for 8 hr for peptization. After the completion of the peptization, the mixture was filtered through a 0.45-μm filter and was subjected to solution exchange with a desalination column (PD-10, manufactured by Amersham Pharmacia Bioscience) to prepare an acidic titanium dioxide sol having a solid content of 1%. This dispersion was placed in a 100-ml vial bottle and was ultrasonicated at 200 Hz for 30 min. The average dispersed particle diameters were 36.4 nm before the ultrasonication and 20.2 nm after the ultrasonication. After the ultrasonication, the solution was concentrated to prepare a titanium dioxide sol having a solid content of 20%. The titanium dioxide sol (0.75 ml) thus obtained was dispersed in 20 ml of dimethylformamide (DMF), followed by the addition of 10 ml of DMF containing 450 mg of polyethyleneimine (average molecular weight: 10000, manufactured by Wako Pure Chemical Industries, Ltd.) dissolved therein. The mixture was then stirred for mixing. The solution was transferred to a hydrothermal reaction vessel (HU-50, manufactured by SAN-AI Science Co. Ltd.), and a synthesis reaction was allowed to proceed at 150° C. for 6 hr. After the completion of the reaction, the reaction solution was cooled to a reaction vessel temperature of 50° C. or below. Isopropanol (manufactured by Wako Pure Chemical Industries, Ltd.) in an amount of twice the amount of the reaction solution was added to the cooled reaction solution. The mixture was allowed to stand at room temperature for 30 min, and the resultant precipitate was collected by centrifugation. The collected precipitate was washed with 70% ethanol, and 2.5 ml of water was added thereto to prepare a dispersion of polyethyleneimine-bonded titanium dioxide particles (anatase-type). The dispersed particle diameter of the polyethyleneimine-bonded titanium dioxide particles was measured with Zetasizer Nano ZS (manufactured by SYSMEX CORPORATION) by charging 0.75 ml of the dispersion of polyethyleneimine-bonded titanium dioxide particles into a zeta potential measuring cell, setting various parameters of the solvent to the same values as those of water, and measuring the dispersed particle diameter at 25° C. by a dynamic light scattering method. As a result, it was found that the average particle diameter of the polyethyleneimine-bonded titanium dioxide particles was 65.6 nm. The zeta potential of the polyethyleneimine-bonded titanium dioxide particles was measured with Zetasizer Nano ZS under the same conditions as described above, and was found to be +35.7 mV.

### Example A2

### Introduction of Polyethyleneimine into Titanium Dioxide Particles (2)

Polyethyleneimine-bonded titanium dioxide particles were synthesized in the same manner as in Example A1, except that polyethyleneimine having an average molecular weight of 7500 was used. As a result, also when the polyethyleneimine having an average molecular weight of 7500 was used, the dispersion of the polyethyleneimine-bonded titanium dioxide particles (anatase-type) exhibited satisfactory dispersibility, thus being suitable.

### Example A3

### Introduction of Polyethyleneimine into Titanium Dioxide Particles (3)

Polyethyleneimine-bonded titanium dioxide particles were synthesized in the same manner as in Example A1, except that an alkaline titanium dioxide sol (Tynoc AL-6, manufactured by Taki Chemical Co., Ltd.) was used instead of the acidic titanium dioxide sol. As a result, also when the alkaline titanium dioxide sol was used, the dispersion of the polyethyleneimine-bonded titanium dioxide particles (anatase form) exhibited satisfactory dispersibility, thus being suitable.

### Example A4

### Introduction of Polyethyleneimine into Magnetic Material/Titanium Oxide Composite Particles

Polyoxyethylene(15) cetyl ether (C-15: manufactured by NIHON SURFACTANT KOGYO K.K.) (45.16 g) was dissolved in a separable flask, and the inside of the flask was purged with nitrogen for 5 min. Thereafter, 75 ml of cyclohexene (manufactured by Wako Pure Chemical Industries, Ltd.) was added to the flask, and 3.6 ml of 0.67 M aqueous solution of FeCl₂ (manufactured by Wako Pure Chemical Industries, Ltd.) was further added. While stirring at 250 rpm, 5.4 ml of 30% aqueous ammonia solution was added to the mixture, and a reaction was allowed to proceed for one hr. Then, 0.4 ml of a 50 mM aqueous tetraethylorthosilicate solution (manufactured by Wako Pure Chemical Industries, Ltd.) was added dropwise to the mixture, and a reaction was allowed to proceed for one hr. Titanium tetraisopropoxide (manufactured by Wako Pure Chemical Industries, Ltd.) was then added to a final concentration of 5 mM, followed by the addition of 10 ml of 50% (w/v) aqueous ethanol solution in 1 ml portions at intervals of 10 min. The aqueous solution was centrifuged, and the resultant precipitate was fired at 350° C. for 2 hr. After the firing, the fired product was dispersed in 10 mM aqueous nitrate solution, and the dispersion was ultrasonicated and was then filtered through a 0.1-μm filter. The magnetic material/titanium oxide composite sol (0.75 ml) thus obtained was dispersed in 20 ml of dimethylformamide (DMF). DMF (10 ml) containing polyethyleneimine (average molecular weight: 10000, manufactured by Wako Pure Chemical Industries, Ltd.) (450 mg) dissolved therein was added to the dispersion. The mixture was then stirred for mixing. The solution was transferred to a hydrothermal reaction vessel (HU-50, manufactured by SAN-AI Science Co. Ltd.), and a synthesis reaction was allowed to proceed at 150° C. for 6 hr. After the completion of the reaction, the reaction solution was cooled to a reaction vessel temperature of 50° C. or below. Isopropanol (manufactured by Wako Pure Chemical Industries, Ltd.) in an amount of twice the amount of the reaction solution was added to the reaction solution. The mixture was allowed to stand at room temperature for 30 min and was then centrifuged to collect precipitates. The collected precipitates were washed with 70% ethanol. Water (2.5 ml) was added to the mixture to prepare a dispersion of polyethyleneimine-bonded magnetic material/titanium dioxide composite particles (anatase-type). The dispersion thus obtained did not cause white turbidity and had the particles well-dispersed in the dispersion, thus being satisfactory as in the case of single titanium dioxide.

### Example A5

### Solubility of Polyethyleneimine-Bonded Titanium Dioxide Particles in Isopropanol

A solution of 200 mg of polyethyleneimine dissolved in 10 ml of DMF was designated as a solution (A). A dispersion of 0.25 ml of the titanium dioxide sol having a solid content of 20% prepared in the process in Example A1 in 10 ml of DMF was designated as a solution (B). A mixture of 0.25 ml of the titanium dioxide sol having a solid content of 20% prepared in the process of Example A1 and a solution of 200 mg of polyethyleneimine, which are dissolved in 10 ml of DMF, was designated as a solution (C). A dispersion of polyethyleneimine-bonded titanium dioxide particles prepared by allowing the solution (C) to react at 150° C. for 6 hr was designated as a solution (D). To each of the solutions (A) to (D) was added isopropanol in an amount of twice the amount of the solution. Each of the mixtures was stirred, was allowed to stand, and was then observed for precipitation formation. As a result, it was found that all the solutions (A) to (C) formed a transparent dispersion in isopropanol without precipitate formation, while only the solution (D) formed precipitates. This fact suggests that, as compared with the case where polyethyleneimine was merely mixed with titanium dioxide particles, polyethyleneimine-bonded titanium dioxide particles have stronger bonds between the polyethyleneimine and the titanium dioxide particles so as to form precipitates as affected by isopropanol.

### Example A6

### Stability of Polyethyleneimine-Bonded Titanium Dioxide Particles in Neutral Solution

Each of the solutions respectively having the same compositions as the solutions (A) to (D) prepared in Example A5 was provided and was evaluated for stability in an neutral solution. Specifically, each of the solutions (A) to (D) was diluted with a 200 mM phosphate buffer solution (pH 7.0) by a factor of 10. The diluted solutions were stirred and were then allowed to stand for observation of precipitate formation. As a result, it was found that the solutions (B) and (C) containing the titanium dioxide sol caused precipitate formation while the solutions (A) and (D) did not cause precipitate formation. It is considered that, since the isoelectric point of titanium dioxide is in an approximately neutral pH value, titanium dioxide disadvantageously aggregates in the solutions (B) and (C), causing precipitates. On the other hand, for the solution (D), since the titanium dioxide surface is modified with innumerable amines, the whole particles are positively charged at an approximately neutral pH value, retaining the solution in a homogeneously dispersed state. Specifically, in the comparison of the solution (C) with the solution (D), the results of Examples A7 and A8 show that the polyethyleneimine-bonded titanium dioxide particles exhibit properties completely different from those of the titanium dioxide particles having dispersibility merely enhanced by the addition of polyethyleneimine.

### Example A7

### Determination of Content of Titanium Dioxide in Dispersion of Polyethyleneimine-Bonded Titanium Dioxide Particles

The dispersion of polyethyleneimine-bonded titanium dioxide particles prepared in Example A1 was heat dried at 110° C. for one hr and was further ignited for 4 hr for complete incineration. The ash thus obtained was cooled in a silica gel desiccator, and the mass of the cooled ash was measured as the net content of titanium dioxide in the dispersion. As a result, it was found that the content of titanium dioxide in the dispersion was 0.25% (w/v).

### Example A8

### Determination of Content of Amino Group in Dispersion of Polyethyleneimine-Bonded Titanium Dioxide Particles

Confirmation and quantitative determination of the amino group in the polyethyleneimine-bonded titanium dioxide particles prepared in Example A1 were carried out through a reaction of the amino group with fluorescamine (manufactured by Tokyo Chemical Industry Co., Ltd.). Since fluorescamine can react with the amino group to produce a fluorescent substance, the confirmation and quantitative determination of the amino group can be carried out by measuring the fluorescence intensity of a reaction product between the polyethyleneimine-bonded titanium dioxide particles and fluorescamine. A glucosamine solution having a predetermined concentration was prepared using a 100 mM borate buffer solution (pH 9.0), and a calibration line was prepared for fluorescence intensity under conditions of excitation wavelength 395 nm and fluorescence wavelength 480 nm. The content of the amino group on the polyethyleneimine-bonded titanium dioxide particle was determined using this calibration line. As a result, it was suggested that the concentration of the amino group in the above dispersion was 4.01×10⁻² M. Based on the results of Example A7, the amino group/titanium dioxide content ratio in the dispersion was 1.63×10⁻² (mol/g).

### Example A9

### Evaluation on Photocatalytic Activity of Polyethyleneimine-Bonded Titanium Dioxide Particles (Anatase-Type)

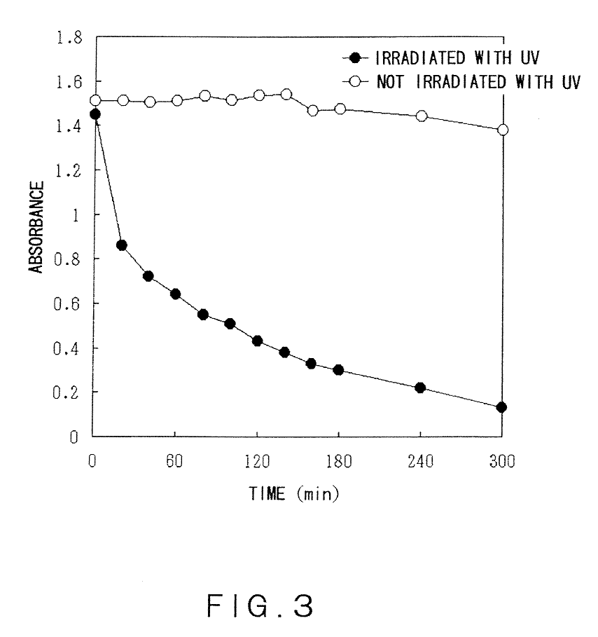

The polyethyleneimine-bonded titanium dioxide particles (anatase-type) prepared in Example A1 were diluted with a 50 mM phosphate buffer solution (pH 7.0) to a solid content of 0.02%. Methylene blue trihydrate (manufactured by Wako Pure Chemical Industries, Ltd.) was added to the aqueous solution to a concentration of 40 μM. The aqueous solution was irradiated with ultraviolet ray with a wavelength of 340 nm at an exposure of 1.5 mW/cm² with stirring, and the absorption at a wavelength of 580 nm was measured with an ultraviolet-visible spectrophotometer. The results are shown in FIG. 3. As compared with the mixed liquid not irradiated with ultraviolet ray, the mixed liquid irradiated with ultraviolet light exhibited a reduction in absorbance due to decomposition of methylene blue with the elapse of the irradiation time, clearly indicating that the polyethyleneimine-bonded titanium dioxide particles (anatase-type) prepared in Example A1 had photocatalytic activity.

### Example A10

### Evaluation on Stability of Polyethyleneimine-Bonded Titanium Dioxide Particles against pH Values

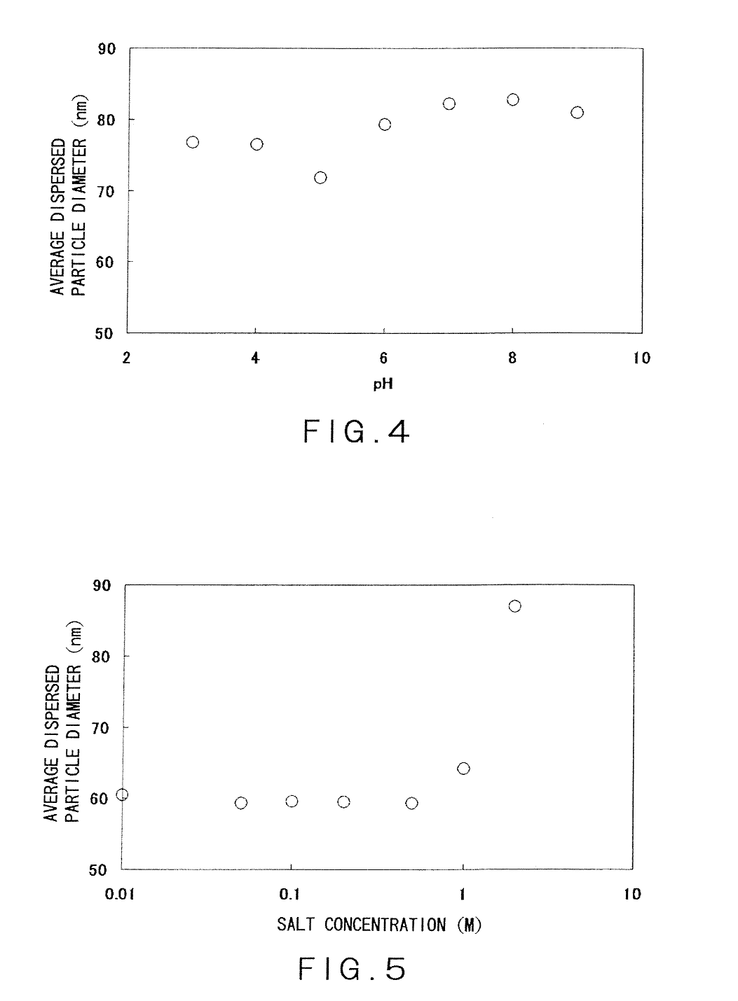

Buffer solutions (50 mM) having different pH values (pH 3=glycine hydrochloride buffer solution, pH 4 and 5=acetate buffer solution, pH 6=2-morpholinoethanesulfonate buffer solution, pH 7 and 8=2-[4-(2-hydroxyethyl)-1-piperazinyl]ethanesulfonate buffer solution, pH 9=borate buffer solution, and pH 10=glycine sodium hydroxide buffer solution) were prepared. The polyethyleneimine-bonded titanium dioxide particle-containing dispersion prepared in Example A1 was added to the buffer solutions to a final concentration of 0.025 (w/v) %. The mixtures were allowed to stand at room temperature for one hr. The average dispersed particle diameter was then measured with Zetasizer Nano ZS in the same manner as in Example A1. The results are shown in FIG. 4. Although there was a change in particle diameter between pH 3 and pH 10, the particle diameter was about 70 to 85 nm and the dispersibility was stable.

### Example A11

### Evaluation on Stability of Polyethyleneimine-Bonded Titanium Dioxide Particles against Salt Strength Values

Polyethyleneimine-bonded titanium dioxide particles prepared in Example A1 was added to 10 mM phosphate buffer solutions containing sodium chloride in different concentrations of 0.05 to 5 M to a final concentration of 0.025%, and the mixtures were allowed to stand at room temperature for one hr. The average dispersed particle diameter was then measured with Zetasizer Nano ZS in the same manner as in Example A1. The results are shown in FIG. 5. There was substantially no change in average dispersed particle diameter in the range of 0.05 to 1 M of salt concentration in the system, showing stable dispersibility.

### Example A12

### Evaluation on Homogeneity (Transparency) of Titanium Dioxide Composite Particles

The polyethyleneimine-bonded titanium dioxide particle-containing dispersion prepared in Example A1 was diluted with a 10 mM phosphate buffer solution containing 0.1 M sodium chloride to a final concentration of 0.1%, and the mixture was allowed to stand at room temperature for one hr. Separately, titanium dioxide particles P25 (manufactured by Nippon Aerosil Co., Ltd.) were diluted with a 10 mM phosphate buffer solution containing 0.1 M sodium chloride to a final concentration of 0.1% in the same manner as described above, and the mixture was allowed to stand at room temperature for one hr. The diluted solutions were then transferred respectively to 5-ml Petri dishes, and were photographed from above the dishes for observation. The results are shown in FIG. 6. It is apparent that, as compared with the aqueous P25 solution, the polyethyleneimine-bonded titanium dioxide particle-containing dispersion had higher transparency and exhibited homogeneous dispersion. Further, the absorbance was measured at a wavelength of 660 nm with a spectrophotometer (UV-1600, manufactured by Shimadzu Seisakusho Ltd.). As a result, the absorbance of the aqueous P25 solution was much higher than 1, which was immeasurable, while the polyethyleneimine-bonded titanium dioxide particle-containing dispersion had an absorbance of 0.044 without forming precipitation. Further, these solutions were allowed to stand in a dark place at room temperature for 2 weeks, followed by the measurement of the absorbance at a wavelength of 660 nm in the same manner as described above. As a result, the absorbance of the aqueous P25 solution was much higher than 1, which was immeasurable, while the dispersion containing the polyethyleneimine-bonded titanium dioxide particles had an absorbance of 0.051. In view of these, it was found that the dispersion of the titanium dioxide composite particles had high transparency, homogeneous dispersibility, and stability, in the aqueous solution.

### Example A13

### Evaluation on Cytotoxity of Polyethyleneimine Titanium Dioxide Particles

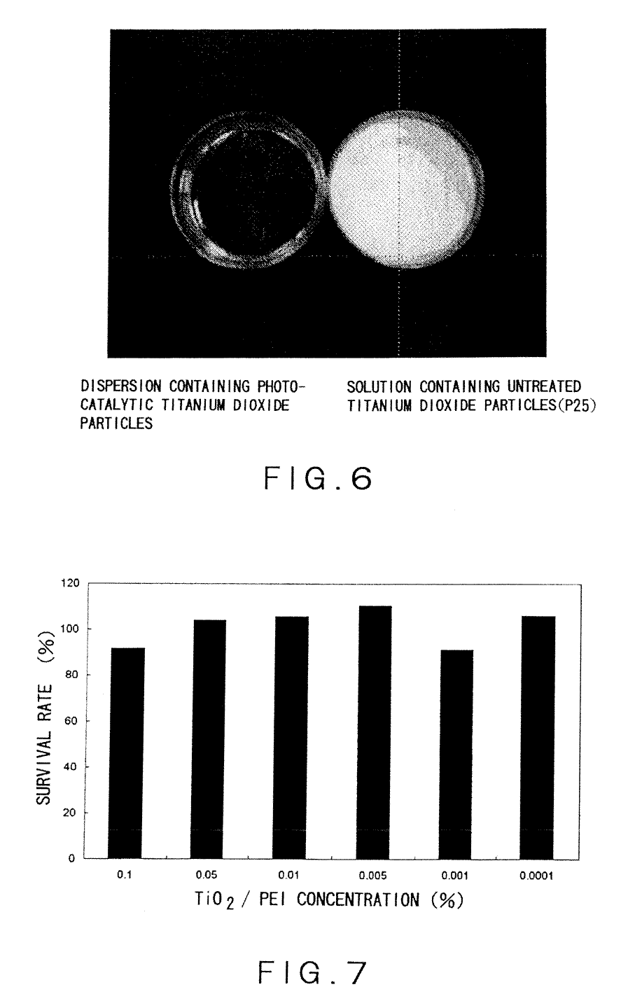

The dispersion containing polyethyleneimine-bonded titanium dioxide particles prepared in Example A1 was diluted with a 10% serum-containing RPMI 1640 medium (manufactured by GIBCO) to a solid content of 1.0%. Culture cancer cells (Jurkat) were cultured in a 10% serum-containing RPMI 1640 medium (manufactured by GIBCO) at 37° C. under a 5% carbon dioxide atmosphere to prepare a cell culture having a concentration of 5.0×10⁴ cells/ml. This cell culture was again cultured for 20 hr under the same conditions. This cell culture was diluted with the polyethyleneimine-bonded titanium dioxide particle-containing dispersion to final concentrations of 0.1%, 0.01%, 0.001%, and 0.0001% on a 96-hole plate to prepare a 200-μl cell culture for a test. This cell culture for a test was cultured at 37° C. under a 5% carbon dioxide atmosphere for 20 hr. Each culture (100 μl) was subjected to a viable cell-derived luminous reaction by Celltiter-Glo Luminescent Cell Viability Assay (manufactured by Promega). The cytotoxity was evaluated by measuring the luminescence level with an image analyzer LAS-3000 UVmini (manufactured by Fuji Photo Film Co., Ltd.). The results are shown in FIG. 7. As compared with the luminescence level in culture cells for a control to which any substance had not been added, all the dispersion concentrations resulted in substantially identical luminescence levels, indicating that the dispersion containing polyethyleneimine-bonded titanium dioxide particles in this concentration range had no cytotoxity.

### Example A14

### Evaluation on Cell Uptake Property of Polyethyleneimine-Bonded Titanium Dioxide Particles

The titanium dioxide sol of 0.75 ml prepared in Example A1 was dispersed in 20 ml of dimethylformamide (DMF). A solution of 0.2 g of polyacrylic acid (average molecular weight: 5000, manufactured by Wako Pure Chemical Industries, Ltd.) in 10 ml of DMF was added to the dispersion, followed by mixing with stirring. The solution was transferred to a hydrothermal reaction vessel, and hydrothermal synthesis was allowed to proceed at 180° C. for 6 hr. After the completion of the reaction, the reaction solution was cooled to a reaction vessel temperature of 50° C. or below. The cooled reaction solution was taken out of the reaction vessel. Water (80 ml) was added to and mixed with the cooled reaction solution while stirring. DMF and water were removed by an evaporator. Water (20 ml) was again added to the mixture to prepare an aqueous solution of polyacrylic acid-bonded titanium dioxide particles. Hydrochloric acid (2 N, 1 ml) was added to the aqueous solution to precipitate titanium dioxide particles. The system was centrifuged, and the supernatant was removed to separate polyacrylic acid remaining unreacted. Water was again added for washing, and the mixture was centrifuged to remove water. A 50 mM phosphate buffer solution (pH 7.0, 10 ml) was added, and the mixture was ultrasonicated at 200 Hz for 30 min to disperse titanium dioxide particles. After the ultrasonication, the dispersion was filtered through a 0.45-μm filter to provide a dispersion of 0.25 wt % polyacrylic acid-bonded titanium dioxide particles. The average particle diameter of the polyacrylic acid-bonded titanium dioxide particles thus obtained was measured with Zetasizer Nano ZS (manufactured by SYSMEX CORPORATION) by charging 0.75 ml of the dispersion of polyethyleneimine-bonded titanium dioxide particles into a zeta potential measuring cell, setting various parameters of the solvent to the same values as those of water, and measuring the dispersed particle diameter at 25° C. by a dynamic light scattering method. As a result, it was found that the average particle diameter of the polyacrylic acid titanium dioxide particles was 45.9 nm. 1-Ethyl-3-[3-dimethylaminopropyl]carbodiimide hydrochloride (0.8 M, 250 μl) and N-hydroxysuccinimide (250 μl) were added to 2 ml of the dispersion of polyacrylic acid-bonded titanium dioxide particles. The reaction solution was allowed to react at room temperature with stirring for one hr. The mixture was subjected to gel filtration for solution exchange through a desalination column (NAP-10, manufactured by Amersham Pharmacia Bioscience) equilibrated with a 10 mM acetate buffer solution (pH 5.0), and the total amount of the solution was brought to 9.5 ml with a 10 mM acetate buffer solution (pH 5.0). A 100 mM solution (5 μl) of 5-amino fluorescein (manufactured by NCI) dissolved in DMF was added to the mixture, and the mixture was allowed to react under light shielding conditions at room temperature with stirring for one hr. Next, 500 μl of an aqueous solution of 0.1 M ethanolamine (manufactured by Wako Pure Chemical Industries, Ltd.) was added to the mixture, and the mixture was allowed to react with stirring at room temperature under light shielding conditions for 30 min. This solution was subjected to gel filtration through a desalination column PD-10 equilibrated with 100 mM phosphate buffer brine (pH 7.5) for solution exchange, and 5-amino fluorescein remaining unreacted was then separated. The solution was then concentrated to 2 ml to obtain a dispersion of fluorescent dye-labeled polyacrylic acid-bonded titanium dioxide particles.

Separately, 500 μl of the dispersion of polyethyleneimine-bonded titanium dioxide particles prepared in Example A1 was subjected to gel filtration through a desalination column NAP-10 equilibrated with a 100 mM phosphate buffer brine (pH 7.5) for solution exchange, and fluorescein isothiocyanate (manufactured by Pierce) dissolved in DMSO to a final concentration of 0.8 mM was added to the mixture, and the mixture was gently stirred at room temperature for 30 min. After the completion of the reaction, the solution was subjected to solution exchange through PD-10 (manufactured by Amersham Pharmacia Bioscience) which had been previously equilibrated with PBS. The solution was then concentrated to 2 ml to obtain a dispersion of fluorescent substance-labeled polyethyleneimine-bonded titanium dioxide particles.

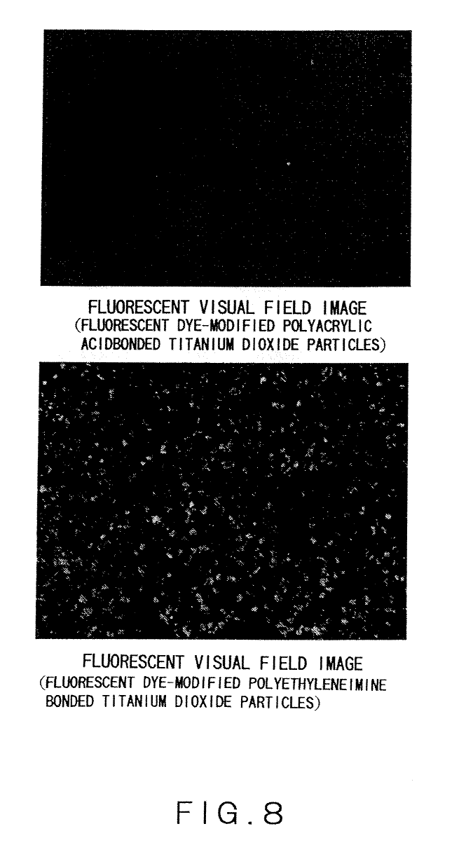

Next, a melanoma cell line T-24 was cultured in a 10% serum-containing F12 medium (manufactured by Gibco) until 100% confluent. The flask was washed twice with 100 mM phosphate buffer brine (pH 7.4). A 100 mM trypsin-ethylenediamine triacetic acid solution (1 ml) was added to the flask, and was allowed to stand for 10 min. The cells peeled from the wall surface of the flask were recovered and were diluted with an F12 medium containing 9 ml of 10% serum. The number of cells were counted with a haemocytometer. The medium containing 5×10⁴ cells (500 μl) was inoculated in a 24-hole microtiter plate and was dispensed so that the final concentration was 0.01%. The dispersion of fluorescent dye-labeled polyacrylic acid-bonded titanium dioxide particles and the dispersion of fluorescent dye-labeled polyethyleneimine-bonded titanium dioxide particles were added respectively in an amount of 100 μl so that the final concentration was 0.01%, and the mixture was cultured within a CO₂ incubator for 24 hr. The adhesion of cells to the flask was then confirmed, and the flask was washed with 100 mM phosphate buffer brine. An F12 medium containing 10% serum (200 μl) was added to the flask, and was observed through a fluorescence microscope to obtain an image shown in FIG. 8. As a result of observation of a fluorescent visual image, it was confirmed that the fluorescent dye-labeled polyethyleneimine-bonded titanium dioxide particles clearly had higher cell affinity and higher cell uptake property than the fluorescent dye-labeled polyacrylic acid-bonded titanium dioxide particles.

### Example A15

### Evaluation of Cell Killing Properties of Polyethyleneimine Titanium Dioxide Particles

A melanoma cell line T-24 was cultured in a 10% serum-containing F12 medium (manufactured by Gibco) until 100% confluent. The flask was washed twice with 100 mM phosphate buffer brine (pH 7.4). A 100 mM trypsin-ethylenediamine triacetic acid solution (1 ml) was added to the flask, and was allowed to stand for 10 min. The cells peeled from the wall surface of the flask were recovered and were diluted with an F12 medium containing 9 ml of 10% serum. The number of cells were counted with a haemocytometer. The medium containing 5×10⁴ cells (500 μl) was inoculated in a 24-hole microtiter plate. The dispersion of polyethyleneimine-bonded titanium dioxide particles prepared in Example A1 was diluted with 100 mM phosphate buffer brine (pH 7.4), and 100 μl of the diluted solution was added to the titer plate so that the final concentrations were 0% and 0.01%, respectively. Ultraviolet ray with a wavelength of 340 nm was applied from a blacklight (manufactured by Toshiba Corp.) to the titer plate at 2.5 mW/cm² for 0 min and 60 min, followed by culture within a CO₂ incubator for 24 hr. A cell counting kit-8 (manufactured by DOJINDO LABORATORIES) was prepared according to an instruction manual for a reagent and was added to the culture, and the absorbance was measured at a wavelength of 450 nm on an 96-hole plate with an absorptiometer (Benchmark, manufactured by Bio-Rad). The results are shown in Table 1.

TABLE 1

| | UV irradiation | UV irradiation | UV irradiation | UV irradiation |
|---|---|---|---|---|
| | time 0 min | time 60 min | time 0 min | time 60 min |
| Experimental | Titanium dioxide | Titanium dioxide | Titanium dioxide | Titanium dioxide |
| condition | concentration 0% | concentration 0% | concentration 0.01% | concentration 0.01% |
| Relative | 1 | 0.9 | 1.2 | 0.4 |
| survival rate | | | | |

The relative survival rate was determined in view of 1 being the viable cell-derived absorbance under conditions of ultraviolet ray irradiation from which a background value was subtracted for 0 (zero) min and a polyethyleneimine-bonded titanium dioxide particle concentration of 0 (zero) %. The results show that the relative survival rate is reduced only when experimental conditions were such that the content of polyethyleneimine-bonded titanium dioxide particles was 0.01% and the ultraviolet ray irradiation time was 60 min, indicating that the polyethyleneimine titanium dioxide particles had high cell killing properties.

### Example B1

### Introduction of Polyethyleneimine into Titanium Dioxide

Titanium tetraisopropoxide (3.6 g) was mixed with 3.6 g of isopropanol, and the mixture was added dropwise to 60 ml of ultrapure water under ice cooling for hydrolysis. After the dropwise addition, the mixture was stirred at room temperature for 30 min. After the stirring, 1 ml of 12 N nitric acid was added dropwise to the mixture, and the mixture was stirred at 80° C. for 8 hr for peptization. After the completion of the peptization, the mixture was filtered through a 0.45-μm filter and was subjected to solution exchange with a desalination column (PD10, manufactured by Amersham Pharmacia Bioscience) to prepare an acidic titanium dioxide sol having a solid content of 1%. This dispersion was placed in a 100-ml vial bottle and was ultrasonicated at 200 Hz for 30 min. The average dispersed particle diameters were 36.4 nm before the ultrasonication and 20.2 nm after the ultrasonication. After the ultrasonication, the solution was concentrated to prepare a titanium dioxide sol having a solid content of 20%. The titanium dioxide sol (0.75 ml) thus obtained was dispersed in 20 ml of dimethylformamide (DMF), and 10 ml of DMF containing 450 mg of polyethyleneimine (average molecular weight: 10000, manufactured by Wako Pure Chemical Industries, Ltd.) dissolved therein was added to the dispersion, followed by stirring for mixing. The solution was transferred to a hydrothermal reaction vessel (HU-50, manufactured by SAN-AI Science Co. Ltd.), and a synthesis reaction was allowed to proceed at 150° C. for 6 hr. After the completion of the reaction, the reaction solution was cooled to a reaction vessel temperature of 50° C. or below. Isopropanol in an amount of twice the amount of the reaction solution was added to the solution to precipitate polyethyleneimine-bonded titanium dioxide particles. After centrifugation, the supernatant was removed to separate polyethyleneimine remaining unreacted. To the residue was added 70% ethanol for washing. After centrifugation, ethanol was removed. Distilled water (10 ml) was added, and the mixture was ultrasonicated at 200 Hz for 30 min to disperse polyethyleneimine-bonded titanium dioxide particles. After the ultrasonication, the mixture was filtered through a 0.45-μm filter to prepare a dispersion of polyethyleneimine-bonded titanium dioxide particles having a solid content of 1.5%. The dispersed particle diameter of the polyethyleneimine-bonded titanium dioxide particles was measured with Zetasizer Nano ZS (manufactured by SYSMEX CORPORATION) by charging 0.75 ml of the dispersion of polyethyleneimine-bonded titanium dioxide particles into a zeta potential measuring cell, setting various parameters of the solvent to the same values as those of water, and measuring the dispersed particle diameter at 25° C. by a dynamic light scattering method. As a result, it was found that the average particle diameter of the polyethyleneimine-bonded titanium dioxide particles was 67.7 nm.

### Example B2

### Immobilization of Protein onto Polyethyleneimine-Bonded Titanium Dioxide Particles

A mixed liquid (0.1 ml) composed of 20 mM 1-ethyl-3-(3-dimethylaminopropyl)carbodiimide (EDC) and 5 mM N-hydroxysuccinimide (NHS) was added to 1 ml of a 50 mM 2[4-(2-hydroxyethyl)-1-piperazinyl]ethanesulfonic acid (HEPES) buffer solution (pH 8.0) containing 0.1 mg of streptavidin (manufactured by Pierce), and the mixture was stirred for 5 min to activate the carboxyl group in streptavidin. After the completion of the stirring, the mixture was subjected to gel filtration with a desalination column NAP-10 (manufactured by Amersham Pharmacia Bioscience) equilibrated with a 10 mM acetate buffer solution (pH 5.0) to remove unreacted EDC and NHS. A streptavidin-containing solution (0.1 ml) was added to 2 ml of the dispersion containing polyethyleneimine-bonded titanium dioxide particles prepared in Example B 1, and the mixture was gently stirred at 4° C. for 10 min. This solution was transferred to a dialysis tube (cut-off molecular weight: 100000, manufactured by Pierce), and dialysis was carried out for 12 hr against a 20 mM tris-hydroxymethylaminomethane-hydrochloride buffer solution (pH 8.0). The solution within the dialysis tube was recovered, and isopropanol in an amount of twice the amount of the solution was added to the solution. The mixture was centrifuged at 4000 g for 10 min. The resultant precipitate was washed with 70% ethanol and was then dissolved in 1 ml of 100 mM phosphate buffer brine (pH 7.5, manufactured by NIPPON GENE CO., LTD.). Thus, a dispersion of photocatalytic titanium dioxide composite particles with streptavidin immobilized thereon was prepared. The diameter of the dispersion was measured with Zetasizer Nano ZS (manufactured by SYSMEX CORPORATION) by charging 0.75 ml of the dispersion into a zeta potential measuring cell, setting various parameters of the solvent to the same values as those of water, and measuring the dispersed particle diameter at 25° C. by a dynamic light scattering method. As a result, it was found that the average particle diameter of the photocatalytic titanium dioxide composite particles was 68.2 nm.

### Example B3

### Confirmation of Biodegradation Capability of Photocatalytic Titanium Dioxide Composite Particles with Streptavidin Immobilized Thereon

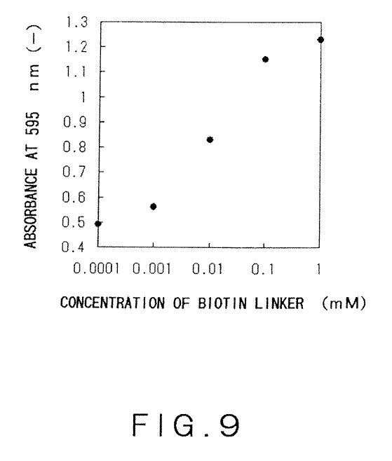

A biotin dimer (EZ-Link PEO-BIOTIN Dimer, manufactured by Pierce) (0.1 ml), which had been diluted to 1 mM to 100 nM by an increment of 10 times, was added to 0.1 ml of the dispersion of streptavidin-immobilized photocatalytic titanium dioxide composite particles prepared in Example B2. The mixture was allowed to stand at 37° C. for 10 min, and the absorbance at 595 nm was measured with a microtiter plate reader (Bench Mark, manufactured by Bio-Rad). The results are shown in FIG. 9. It was found that the turbidity of the solution obviously increased with the concentration of the biotin dimer, demonstrating that streptavidin was efficiently immobilized on the photocatalytic titanium dioxide particles.

### Example B4

### Immobilization of Lectin on Polyethyleneimine-Bonded Titanium Dioxide Particles

The dispersion of polyethyleneimine-bonded titanium dioxide particles prepared in Example B1 was suspended in a 30 mM acetate buffer solution (pH 5.5) to a concentration of 1 (w/v) %. A 500 mM aqueous EDC solution (250 μl) and a 1 mg/ml DBA (Dolichos Biflorus Agglutinin)-FITC (manufactured by VEC: molar bonding ratio of FITC to DBA=2.5) were added to 10 ml of the suspension, and the mixture was stirred at room temperature for 2 hr. After the completion of the reaction, 20 ml of isopropanol was added, and the mixture was allowed to stand at room temperature for 30 min and was then centrifuged at 4000 g for 20 min. The resultant precipitate was washed with 70% ethanol and was suspended in a PBS buffer solution to prepare a dispersion of DBA-FITC-immobilized polyethyleneimine-bonded titanium dioxide particles. The average dispersed particle diameter of these composite particles was 68.3 nm. Fluorescein (manufactured by Wako Pure Chemical Industries, Ltd.) was diluted with a PBS buffer solution, and the diluted solution was measured with a fluorophotometer under conditions of excitation wavelength 595 nm and fluorescence wavelength 625 nm to prepare a calibration line. It was found from the intensity of fluorescence of the dispersion that 600 ng/ml of FITC was bonded. Further, this dispersion was heated to 400° C., and the content of titanium oxide was measured and was found to be 1 (w/v) %. Since DBA:FITC bond ratio was 1:2.5, the DBA-FITC amount was 2.5×10⁻⁷ mol/TiO₂ (g).

### Example B5

### Evaluation of Homogeneity (Clarity) of Dispersion Containing Photocatalytic Titanium Dioxide Composite Particles

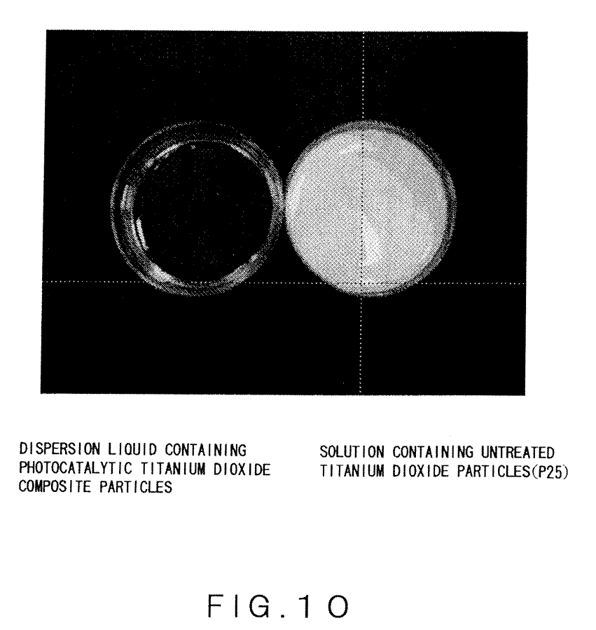

The dispersion containing streptavidin-immobilized photocatalytic titanium dioxide composite particles prepared in Example B2 was diluted with a 0.1 M sodium chloride-containing 10 mM phosphate buffer solution to a final concentration of 0.1%, and the mixture was allowed to stand at room temperature for one hr. Separately, titanium oxide particles P25 (manufactured by Nippon Aerosil Co., Ltd.) were diluted with a 10 mM phosphate buffer solution containing 0.1 M sodium chloride to a final concentration of 0.1% in the same manner as described above, and the mixture was allowed to stand at room temperature for one hr. The diluted solutions were then transferred respectively to 5-ml Petri dishes, and were photographed from above the dishes for observation. The results are shown in FIG. 10. It is apparent that, as compared with the aqueous P25 solution, the dispersion containing streptavidin-immobilized photocatalytic titanium dioxide composite particles had higher transparency and exhibited homogeneous dispersion. Further, the absorbance was measured at a wavelength of 660 nm with a spectrophotometer (UV-1600, manufactured by Shimadzu Seisakusho Ltd.). As a result, the absorbance of the aqueous P25 solution was much higher than 1, which was immeasurable, while the dispersion containing streptavidin-immobilized photocatalytic titanium dioxide composite particles had an absorbance of 0.044 without forming precipitation. Further, these solutions were allowed to stand in a dark place at room temperature for 2 weeks. The absorbance at a wavelength of 660 nm was then measured in the same manner as described above. As a result, the absorbance of the aqueous P25 solution was much higher than 1, which was immeasurable, while the dispersion containing streptavidin-immobilized photocatalytic titanium dioxide composite particles had an absorbance of 0.051. The results show that the dispersion of the titanium dioxide composite particles had high transparency, homogeneous dispersibility, and stability, in the aqueous solution,

### Example B6

### Evaluation of Cell Killing Properties of Dispersion Containing Photocatalytic Titanium Dioxide Composite Particles

A melanoma cell line T-24 was cultured in a 10% serum-containing F12 medium (manufactured by Gibco) until 100% confluent. The flask was washed twice with 100 mM phosphate buffer brine (pH 7.4). A 100 mM trypsin-ethylenediamine triacetic acid solution (1 ml) was added to the flask. The mixture was allowed to stand for 10 min, and the cells peeled from the wall surface of the flask were recovered and were diluted with an F12 medium containing 9 ml of 10% serum. The number of cells were counted with a haemocytometer. The medium containing 5×10⁴ cells (500 μl) was inoculated in a 24-hole microtiter plate. The dispersion containing streptavidin-immobilized photocatalytic titanium dioxide composite particles prepared in Example B2 was diluted with 100 mM phosphate buffer brine (pH 7.4), and 100 μl of the diluted solution was added to the titer plate so that the final concentrations were 0% and 0.01%, respectively. Ultraviolet ray with a wavelength of 340 nm was applied from a blacklight (manufactured by Toshiba Corp.) to the titer plate at 2.5 mW/cm² for 0 min and 60 min, followed by culture within a CO₂ incubator for 24 hr. A cell counting kit-8 (manufactured by DOJINDO LABORATORIES) was prepared according to an instruction manual for a reagent and was added to the culture, and the absorbance was measured at a wavelength of 450 nm on an 96-hole plate with an absorptiometer (Benchmark, manufactured by Bio-Rad). The results are shown in Table 2.

TABLE 2

| | UV irradiation | UV irradiation | UV irradiation | UV irradiation |
|---|---|---|---|---|
| | time 0 min | time 60 min | time 0 min | time 60 min |
| Experimental | Titanium dioxide | Titanium dioxide | Titanium dioxide | Titanium dioxide |
| condition | concentration 0% | concentration 0% | concentration 0.01% | concentration 0.01% |
| Relative | 1 | 1 | 1.1 | 0.5 |
| survival rate | | | | |

The relative survival rate was determined in view of 1 being the viable cell-derived absorbance under conditions of ultraviolet ray irradiation from which a background value was subtracted for 0 (zero) min and a streptavidin-immobilized titanium dioxide particle concentration of 0 (zero) %. The results show that the relative survival rate is reduced only when experimental conditions were such that the content of streptavidin-immobilized photocatalytic titanium dioxide composite particles was 0.01% and the ultraviolet light irradiation time was 60 min, indicating that the dispersion containing streptavidin-immobilized photocatalytic titanium dioxide composite particles had high cell killing properties.

Although there have been described what are the present embodiments of the invention, it will be understood that variations and modifications may be made thereto within the scope of the claims appended hereto.

1. A method for killing cells comprising the steps of
administrating photocatalytic titanium dioxide particles to a mammal, wherein the particles comprising photocatalytic titanium dioxide and a cationic hydrophilic polymer modifying surfaces of the photocatalytic titanium dioxide particles, where the hydrophilic polymer is bonded to the photocatalytic titanium dioxide, whereby the titanium dioxide particles are taken up by cells of the mammal, and
irradiating with UV light the cells to kill the cells with photocatalytic degrading capability of the photocatalytic titanium dioxide particles irradiated with the UV light.

2. The method according to claim 1, wherein the hydrophilic polymer is a hydrophilic polymeric amine.

3. The method according to claim 1, wherein the photocatalytic titanium dioxide is in anatase form or rutile form.

4. The method according to claim 1, wherein the particles comprising photocatalytic titanium dioxide have diameters of 2 to 200 nm. 20

5. The method according to claim 1, wherein the photocatalytic titanium dioxide is a titanium dioxide composite comprising a photocatalytic titanium dioxide and a magnetic material.

6. The method according to claim 1, wherein the hydrophilic polymer is a hydrosoluble polymer.

7. The method according to claim 6, wherein the hydrosoluble polymer is selected from a group consisting of polyamino acids, polypeptides, polyamines, and copolymers containing amine units.

8. The method according to claim 6, wherein the hydrosoluble polymer is selected from a group consisting of polyethyleneimines, polyvinylamines, and polyallylamines.

9. The method according to claim 6, wherein the hydrosoluble polymer comprises a polyethyleneimine.

10. The method according to claim 6, wherein the hydrosoluble polymer comprises a copolymer containing a plurality of amine units in its molecule.

11. The method according to claim 1, wherein the photocatalytic titanium dioxide particles have a surface potential of not less than +20 mV.

12. The method according to claim 1, wherein photocatalytic titanium dioxide particles are administrated as a dispersion of the photocatalytic titanium dioxide particles in which the particles are dispersed in an aqueous solvent.

13. The method according to claim 12, wherein the aqueous solvent has a pH value of 3 to 9.

14. The method according to claim 12, wherein the aqueous solvent is a pH buffer solution.

15. The method according to claim 12, wherein the aqueous solvent has a salt concentration of 1 M or less.

16. The method according to claim 15, wherein the aqueous solvent is a physiological saline.

17. The method according to claim 12, containing 0.0001 to 0.1% by weight of the photocatalytic titanium dioxide particles.

18. The method according to claim 12, wherein the photocatalytic titanium dioxide particles are photocatalytic titanium dioxide composite particles having a tissue-derived molecule immobilized onto an amine in the hydrophilic polymer.

19. The method according to claim 18, wherein the photocatalytic titanium dioxide particles are particles having the photocatalytic titanium dioxide at least on a part of surfaces of the particles.

20. The method according to claim 18, wherein the aqueous solvent is a biologically acceptable aqueous solution.

21. The method according to claim 18, wherein the tissue-derived molecule is selected from a group consisting of amino acids, peptides, simple proteins, and conjugated proteins.

22. The method according to claim 21, wherein the simple protein is a lectin.

23. The method according to claim 18, wherein the tissue-derived molecule is selected from a group consisting of nucleosides, nucleotides, and nucleic acids.

24. The method according to claim 18, wherein the tissue-derived molecule is selected from a group consisting of monosaccharides, sugar chains, polysaccharides, and glycoconjugates.

25. The method according to claim 18, wherein the tissue-derived molecule is selected from a group consisting of simple lipids, complex lipids, and liposomes.

26. The method according to claim 18, wherein the amine in the hydrophilic polymer has a fluorescent dye immobilized thereon, instead of the tissue-derived molecule.

27. The method according to claim 1, wherein the cells are cancer cells.

28. The method according to claim 27, wherein the photocatalytic titanium dioxide particles is applied directly to a part affected by a cancer.

29. The method according to claim 27, wherein the photocatalytic titanium dioxide particles is administrated topically to a solid cancer by injection.
